(19) United States
(12) Patent Application Publication
(10) Pub. No.: US 2002/0102301 A1
(43) Pub. Date: Aug. 1, 2002

(54) Pharmaceutical solid self-emulsifying composition for sustained delivery of biologically active compounds and the process for preparation thereof

(76) Inventor: Joseph Schwarz, Richmond Hill (CA)

Correspondence Address: PENNIE AND EDMONDS, 1155 AVENUE OF THE AMERICAS, NEW YORK, NY, 100362711

(21) Appl. No.: 09/482,109

(22) Filed: Jan. 13, 2000

(51) Int. Cl. A61K 9/20; A61K 9/22

(52) U.S. Cl. 424/468; 424/464

(57) ABSTRACT

A composition for a method of sustained delivery of a biologically active material. The composition provides a biologically active material in a pharmaceutically acceptable hydrophobic phase for mixture with the biologically active material. An emulsifier emulsifies the hydrophobic phase in water for the mixture. The composition further includes a sorbent and a water absorbent polymer, the latter providing sustained release of the biologically active material in the emulsion.

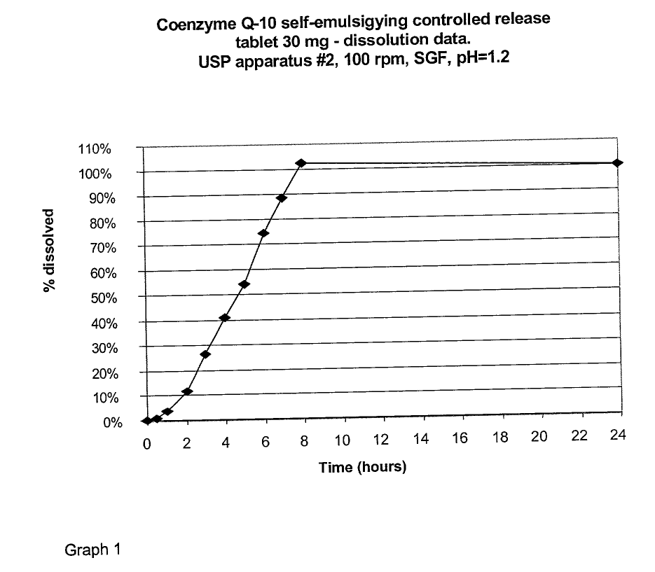

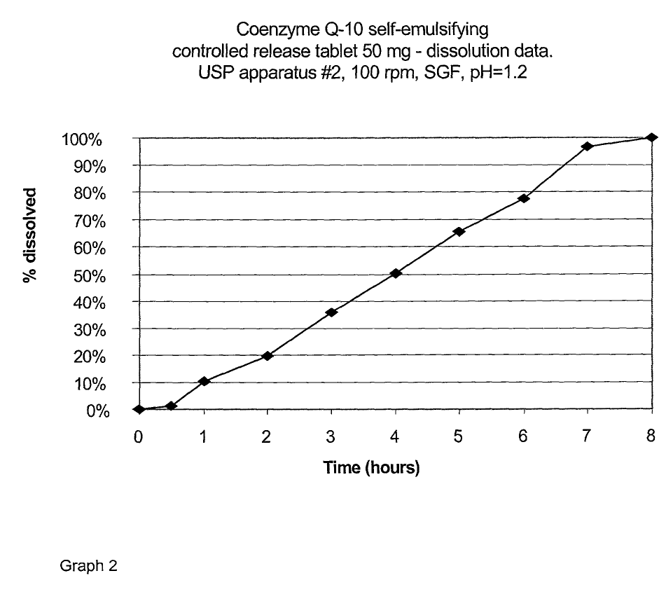

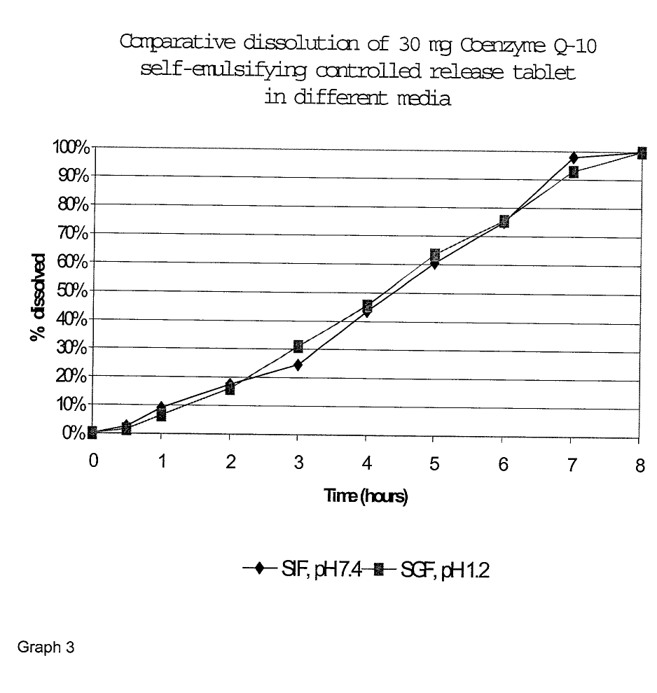

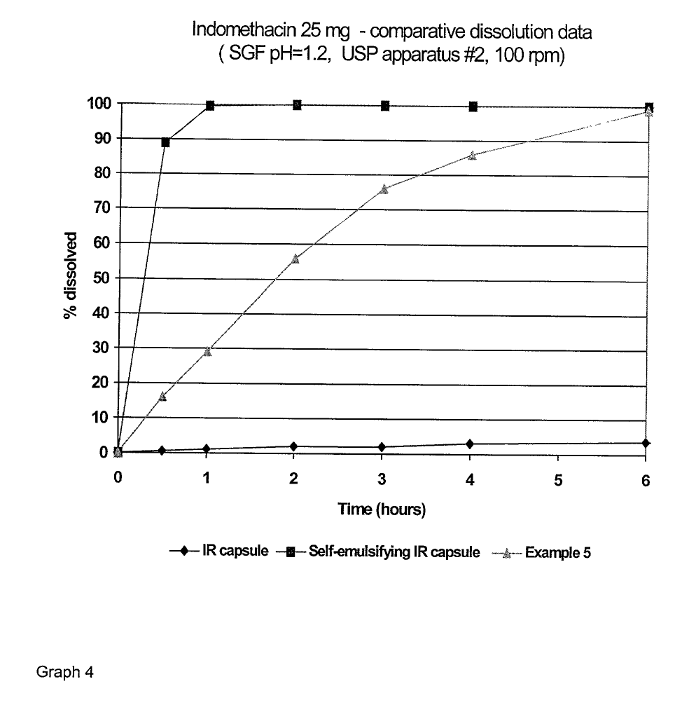

### FIELD OF THE INVENTION

The present invention relates to an extended or prolonged release of medical substances and, more particularly, the present invention relates to a method of oral delivery of hydrophobic drugs with low solubility in water.

### BACKGROUND OF THE INVENTION

The low bio-availability of highly hydrophobic drugs with extremely low water solubility can be a serious problem. Different procedures have been involved in trying to attain a desired level of drug solubility and dissolution rate. These approaches have been based on preparations containing an increased surface area (micronised powders), inclusion complexes (cyclodextrines and derivatives), co-precipitates with water-soluble polymers (PEG, poloxamers, PVP, HPMC) and non-electrolytes (urea, mannitol, sugars etc.), micellar solutions in surfactant systems (Cremophor™, Poloxamers™, Tweens™, Gellucires™), multilayer vesicles (liposomes and niosomes), emulsions, microemulsions and self-emulsifying compositions. Most of these procedures are quite effective for bio-availability improvement of immediate drug release formulations. Moreover, significant increase in bio-availability for such low solublility drugs as nifedipine can lead to dangerous side effects due to dose saturation when using drug solutions in a water miscible vehicle (PEG-400).

Self-emulsifying drug delivery systems are usually comprised of a mixture of the liquid or semi-solid lipid phase (usually fatty acid glycerides or esters), with a surfactant such as oxyethylated glycerides or oxyethylated fatty acids; and additional cosurfactants or cosolvents such as lecithin, monoglycerides, aliphatic alcohols, PEO-PPG copolymers.

A hydrophobic drug can be efficiently dissolved in the mixture. After the addition of water the mixture rapidly converts into an oil-in-water emulsion and, the drug remains in the oil droplets. Absorption of the drug in the gastro-intestinal system through the resulting formed emulsion is markedly increased.

A microemulsion is, to some extent, similar to a self-emulsifying system, and is often comprised of analogous components such as oil, surfactant, short or medium chain alcohol as a cosurfactant, and water, but in another ratio. When diluted with water, an oil-in-water, or water-in-oil emulsion may be produced, according to composition and added water amount. Drug entrapment in minute oil droplets allows for an effective, controlled dispersion of the drug through the lining of the gastro-intestinal tract. Prolonged oral drug delivery is often associated with significantly reduced bio-availability, particularly for compounds with low water solubility.

In the prior art, the preparation of sustained release oral delivery forms has included entrapment of finely dispersed active components into an erodible, water-swellable or lipid-based matrix, coated core tablets, multiparticulate capsules and tablets, porous polymeric sponges, application of a polymeric membrane coating on the sugar particles, and different types of osmotically-driven tablets and capsules (Oros®), Pulsincap®).

### SUMMARY OF THE INVENTION

It has been found that a self-emulsifying microemulsion, bearing a hydrophobic drug, can be successfully placed into a water-swellable polymeric composition, which allows for a sustained release of the hydrophobic drug and, in effect, suspends the rate of release for a desired time interval.

One object of the present invention is to provide a pharmaceutical composition with a sufficient quantity of the low water solubility drug, such that when the drug is dissolved, suspended or dispersed in the self-emulsifying lipid blend, it provides a prolonged release of the included drug from the composition as the drug-bearing, oil-in-water emulsion is gradually released from the composition. To achieve such performance, the self-emulsifying blend is entrapped into a gel-forming, polymeric, water-swellable composition.

In accordance with a further object of one embodiment of the invention there is provided a composition for sustained delivery of a biologically active material, the composition comprising: a biologically active material; a pharmaceutically acceptable hydrophobic phase for mixture with the biologically active material; an emulsifier for providing emulsification of the hydrophobic phase in water; a pharmaceutically acceptable sorbent; and a water absorbing polymer for providing sustained release of the biologically active material in the emulsion.

The self-emulsifying lipid microemulsion may be comprised of: food grade oils and fats, (e.g., soya oil, olive oil, kernel oil, cocoa butter etc); pharmaceutically acceptable glycerides and glycerin fatty acid esters (MCT, tricaprin, trimyristin, triolein and many others); monoglycerides and diglycerides, their mixtures and derivatives (Capful, Miglyol, Myvacet, Witepsol etc.); fatty and aliphatic acids and fatty acid esters (oleic and linoleic acid, ethyl oleate, isopropylmyristate, isopropylpalmitate, isostearic esters, diethyladipate, diethylsebacate etc.); pharmaceutical plastisizers (triethylcitrate, ethyltributylcitrate, dioctylphtalate); lipidic pharmaceutical compounds (tocopherols and their esters, retinol acetate and palmitate, cholesteryl derivatives); phosphatidylcholine derivatives (soy and egg lecithin and analogs); and other compounds which will produce the desired solubility level of the drug-bearing composition.

To achieve the self-emulsifying behavior of the drug-bearing composition, different surfactants or surfactant mixtures having an HLB value, that are adaptable to an oil and drug type, are added in an appropriate ratio. The surfactant may be selected from non-ionic groups such as polyoxyethylated fatty acids (PEG-stearates, PEG-laurates, Brij®); PEG-ethers (Mirj®), sorbitan derivatives (Tweens); aromatic compounds (Tritons®); PEG-glycerides (PECEOL™); PEG-PPG copolymers (Pluronics, Poloxamers, Jeffamines); and polyglycerines, PEG-tocopherols, propylene glycol derivatives, sugar and polysaccharide alkyl and acyl derivatives (octylsucrose, sucrose stearate, lauroyidextran etc.).

Other suitable types of surfactants include anionic surfactants (soaps, sulfonates), cationic surfactants (CTAB), zwitterionic and amphoteric surfactants. The HLB value of the surfactant is dependent mainly on the lipidic composition and, to less extent, on the drug structure.

Lipidic additives, such as fatty acid glycerides, are often used as release retardants for hydrophilic matrices. The hydrophobic nature of these materials decreases the water penetration rate and hydration of the polar polymer and, therefore, reduces the dissolution rate. Another important feature is the lubricating properties of hard glycerides and fatty acids, which are added in relatively small amounts.

Surfactants, mainly non-ionic, are widely suitable to extended release formulations for low solubility drugs. They help to keep the drug in a dissolved or dispersed state, and reduce precipitation by dissolution as they form a micellar solution after contact with water.

The combination of a relatively large quantity of lipidic compount with the correct ratio of a suitable surfactant or surfactant mixture, forms a hydratable polymeric composition, which unexpectedly demonstrates a uniform release of the in-situ formed emulsion with the entrapped drug that dissolves or disperses in the oil phase of the emulsion. An inappropriate ratio between lipid and surfactant leads to formation of an unstable emulsion, or an emulsion that does not form. Similar behavior is observed when the HLB value of the surfactant does not correspond to that of the drug to lipid phase composition. Further, oil droplet size of the formed emulsion may be properly regulated in wide limits to obtain optimal parameters that meet adequate bio-availability and pharmacokinetic guidelines. Droplet size of the “in situ” formed emulsion after matrix hydration can be regulated by appropriate selection of the composition and ratio of the following: the lipidic phase, to the amount and type of drug, to the amount and type of surfactant.

The type and level of excipients added to the self-emulsifying composition is also of high importance. Improper choice of these components leads to weak tablet formation or may make tablet preparation impossible. The emulsification process and the rate of controlled release of the drug may also be affected detrimentally. Accurate selection of the appropriate components is based on consideration of all component properties separately, and in combination.

### DETAILED DESCRIPTION OF THE PREFERRED EMBODIMENTS

The solid self-emulsifying composition for sustained delivery of biologically active compounds is comprised of the following biologically active components: a lipid phase, a surfactant system, a delivery control component, and excipients for tablet formation.

The lipid phase serves as a solvent or vehicle where the active component(s) is dissolved or dispersed. Lipid components may be selected from the following lipid vitamins: alpha-tocopherol, tocopherol acetate, medium chain triglycerides (Miglyol™, Neobee M5); polyol esters (Captex™, lauroyl glycols); acylated glycerides (Myvacet™); food oils (soya, corn, and inert oil); and silicon or paraffin. The lipid phase is selected in accordance with the desired drug solubility in the oil phase, and the stability of formed oil/water emulsions after self-emulsification.

The biocompatible surfactant must have a suitable HLB value and may be selected from the following: polyethoxylated derivatives of tocopherol acid succinate (TPGS™, Eastman-Kodak); glycerides (Gellucire™, Gatefosse); polyol esters (Sorbitan esters, Tween™); sucrose stearates (Sucrose ester™, Gattefosse); long chain acids (PEG stearate, Lipo-PEG™, Mirj™52); or block-copolymers (Poloxamer™, Pluronic™).

The excipients are sorbents which are tablet forming, hydration regulators, that are selected according to desired tablet properties and drug-bearing level. The function of these sorbents is to incorporate the self-emulsifying composition into a free flowing formulation which allows the tablet to form, and holds the lipid composition inside the sorbent during formation of the tablet at high pressure, producing quality tablets with reasonable hardness and friability. Additionally, it prevents drug saturation due to the fast release of active material upon self-emulsification after hydration, and helps achieve homogeneously dispersed material in the tablets. The best sorbents are colloidal silicon dioxide (Syloid™244, Grace) or fumed silica (Aerosil™, Degussa or Cab-O-Sil, Cabot); aluminium silicate (Whithaker); calcium phosphates (Mendell); and microcrystalline cellulose (Avicel™, FMC or Vivapur™).

Release rate control polymers function as the main dissolution rate regulators. After contact with water they form a hydrated gel-like self-emulsified lipid composition. Release of the formed emulsion follows gel dissolution and at least partial diffusion of lipid droplets, from the gelled matrix to the surrounding media. The preferred gel-forming polymers are water swellable or water soluble cellulose derivatives, such as for example, hydroxypropylmethylcellulose (Methocel™, types A, E, K, F, Dow Chemical), hydroxyethylcellulose (Natrosol™, Hercules); hydroxypropylcellulose (Klucel™, Aqualon); and carboxymethylcellulose (cellulose gum). Other types of synthetic polymers include polyacrylic acid (Carbopol™, BFGoodrich); polyethylene oxide (Polyox™, Union Carbide); polyvinylpyrrolidone (Kollidon™, PVP and PVP-VA, BASF); and natural gums and polysaccharides (Xantan gum (Keltrol™, Kelco), carrageenan, locust bean gum, acacia gum, chitosan, alginic acid, hyaluronic acid, pectin, etc.).

Additional additives which may be needed for preparation of the tablet with the desired properties, or for optimization of the filling process for hard capsules are: glidants, lubricants, colors, flavors, sweeteners and taste components, and film coating materials.

The various embodiments of the invention will be explained by way of the following several examples:

### EXAMPLE 1

Coenzyme Q-10 in a Self-Emulsifying Controlled Release Tablet (30 mg strength; dissolution time greater than 6 hours).

As a first example of the first formulation, the slowly dissolving composition contains coenzyme Q-10 (Ubiquinone) in the amount of 30 mg per tablet, is described. The oil phase is comprised of alpha-tocopherol acetate (vitamin E acetate). PEG-40 stearate (Lipo-PEG 39S) has been used as a surfactant with an optimal HLB value for effective emulsification of the oil phase. The weight ratio between Q-10 and the oil phase is 1:1; the surfactant to oil phase w/w ratio is approximately 1.6:1.

The composition of the 30 mg Q-10 self-emulsifying extended release tablet is provided in Table 1.

TABLE 1
Pharmaceutical Solid Self-Emulsifying Composition for
Sustained Delivery of Coenzyme Q-10 (30 mg tablet)

| | Per | % |
|---|---|---|
| | tablet, | By |
| INGREDIENT | mg | Weight |
| Coenzyme Q-10 | 30 | 6.41 |
| Tocopherol acetate | 30 | 6.41 |
| PEG-40 stearate | 50 | 10.68 |
| Colloidal silicon dioxide (Cab-O-Sil) | 15 | 3.21 |
| Dibasic calcium phosphate | 45 | 9.62 |
| Lactose (spray dried) | 110 | 23.50 |
| Methocel E-15 | 24 | 5.13 |
| Methocel K4M | 48 | 10.26 |
| Microcrystalline cellulose (Vivapur pH 102) | 90 | 19.34 |
| PEG 8000 | 18 | 3.85 |
| Povidone (PVP K-25) | 6 | 1.28 |
| Magnesium stearate | 2 | 0.43 |
| Tablet weight: | 468 | 100.00 |

To prepare the composition, coenzyme Q-10, surfactant (PEG stearate), and the oil phase (alpha-tocopherol acetate), were heated together at between 50 and 55° C., then mixed until the coenzyme completely dissolved. This solution was diluted with ethyl-alcohol and mixed with the following sorbents: colloidal silicon dioxide, dibasic calcium phosphate and a quantity of microcrystalline cellulose. The resulting paste was carefully mixed to reach an homogenous dispersion. This dispersion was transferred to a planetary granulator and carefully mixed with the following gel-forming polymers: methocel K4 M, methocel E15, and a quantity of lactose (hydration rate regulator). The mixture was granulated with a separately prepared 5% binder solution of polyvinylpyrrolidone (Kollidon PVP K-25) in ethyl-alcohol, until a suitable granulate was obtained. This granulate was transferred into oven and dried at 45° C. until the volatile solvent evaporated.

The remaining dry granulate was passed through 16 mesh sieve and mixed with microcrystalline cellulose, lactose and sieved magnesium stearate (lubricant). Oval, or capsule-shaped tablets were prepared using regular equipment (16-station rotary tablet press) to yield tablets having a hardness of between 4 and 8 kg, and a friability of less than 1%.

Dissolution tests were performed in accordance with USP requirements, using USP apparatus #2 at 37° C., with a paddle rotation of 100 rpm. A volume of 900 ml of simulated gastric fluid (SGF), or simulated intestinal fluid (SIF), each of which contained no enzymes, served as the dissolution media.

Dissolution was insensitive to media type. The tablet was almost completely dissolved in between 6 and 8 hours. Upon dissolution, the colloidal emulsion of the coenzyme Q-10 which dissolved in the oil phase formed, and was gradually released into the dissolution media, forming a hazy bluish dispersion. The rate of dissolution is displayed on graph 1.

### EXAMPLE 2

Coenzyme Q-10 in a Self-Emulsifying Controlled Release Tablet (50 mg strength) The tablet composition is displayed in table 2.

TABLE 2
Pharmaceutical Solid Self-Emulsifying Composition for
Sustained Delivery of Coenzyme Q-10 (50 mg tablet)

| | Per | % |
|---|---|---|
| | tablet, | By |
| INGREDIENT | mg | Weight |
| Coenzyme Q-10 | 50 | 7.06 |
| Tocopherol acetate | 50 | 7.06 |
| PEG-40 stearate | 80 | 11.30 |
| Colloidal silicon dioxide (Cab-O-Sil) | 25 | 3.53 |
| Dibasic calcium phosphate | 75 | 10.59 |
| Microcrystalline cellulose (Vivapur pH 102) | 125 | 17.65 |
| Methocel K4M | 35 | 4.94 |
| Methocel E-15 | 75 | 10.59 |
| Lactose (spray dried) | 150 | 21.18 |
| Povidone (PVP K-25) | 10 | 1.41 |
| PEG 8000 | 30 | 4.24 |
| Magnesium stearate | 3 | 0.42 |
| Tablet weight: | 708 | 100.00 |

The tablet was prepared as described in Example 1. Tablet hardness was between 6 and 10 kg, with a friability of less than 1%. The rate of dissolution is presented on graph 2.

Drug release from the self-emulsifying matrix is dependent upon the type of media in which the dissolution occurs. Graph 3 represents measurements taken in accordance with USP 23, and shows the rate of dissolution in both acidic and basic conditions, which simulate gastric and intestinal fluids containing no enzymes.

### EXAMPLE 3

Alpha-lipoic Acid in a Self-Emulsifying Controlled Release Tablet (50 mg strength)

This is a slowly dissolving composition containing alpha-lipoic (octathioic) acid in the amount of 50 mg per tablet. The oil phase contains alpha-tocopherol acetate (vitamin E acetate). A derivative of tocopherol, (tocopherol acid succinate PEG1000 ester (TPGS™)) has been used as a surfactant. The weight ratio between lipoic acid and the oil phase is 1:1, with a surfactant to oil phase w/w ratio of 1:1.

The composition of the 50 mg alpha-lipoic acid in a 50 mg self-emulsifying extended release tablet is displayed in table 3.

TABLE 3
Solid Self-Emulsifying Pharmaceutical Composition for
Sustained Delivery Of Alpha-Lipoic Acid (50 mg tablet)

| | Per | % |
|---|---|---|
| | tablet, | By |
| INGREDIENT | mg | Weight |
| alpha-lipoic acid | 50 | 6.41 |
| alpha-Tocopherol acetate | 50 | 6.41 |
| TPGS (PEG1000-tocopherol succinate) | 50 | 10.68 |
| Colloidal silicon dioxide (Cab-O-Sil) | 15 | 3.21 |
| Dibasic calcium phosphate | 45 | 9.62 |
| Lactose (spray dried) | 110 | 23.50 |
| Methocel E-15 | 24 | 5.13 |
| Methocel K4M | 48 | 10.26 |
| Microcrystalline cellulose (Vivapur pH 102) | 90 | 19.34 |
| PEG 8000 | 18 | 3.85 |
| Povidone (PVP K-25) | 6 | 1.28 |
| Magnesium stearate | 2 | 0.43 |
| Tablet weight: | 508 | 100.00 |

Preparation of the composition involves the mixture of alpha-lipoic acid and, alpha-tocopherol acetate, with the surfactant, alpha-tocopherol acid succinate (PEG1000 (TPGS™)). The composition is blended together in dry ethanol until all components are completely dissolved. This solution is then mixed with the following sorbents: colloidal silicon dioxide, dibasic calcium phosphate and a quantity of microcrystalline cellulose. The resultant paste was mixed until homogenous dispersion occurred. The dispersion was then transferred to a suitable granulator and carefully mixed with gel-forming polymers such as: methocel K4M, methocel E15 and a quantity of lactose (hydration rate regulator).

The resulting composition was then granulated with a separately prepared 5% binder solution of polyvinylpyrrolidone (Kollidon PVP K-25) in ethyl-alcohol until a suitable granulate was obtained. This granulate was transferred into an oven and dried at 45° C. until the volatile solvent had totally evaporated.

The remaining dried granulate was passed through a 16 mesh sieve and mixed with microcrystalline cellulose, lactose and sieved magnesium stearate (lubricant). Oval or capsule-shaped tablets were prepared using the 16-station rotary tablet press. The resulting tablet had a hardness of between 5 and 8 kg, and a friability of less than 1%.

Dissolution tests were performed in accordance with USP requirements, using USP apparatus #2 at 37° C., with a paddle rotation of 100 rpm. The tablet was completely dissolved in 6 hours. Upon dissolution, a colloidal emulsion of oil droplets is formed and is gradually released into the dissolution media, forming a hazy bluish dispersion. The active ingredient, alpha-lipoic acid, was distributed between oil droplets in the water phase in accordance with the partition coefficient and pH of the dissolution media.

The rate of dissolution is similar to that in Examples 1 and 2.

Example 4

Indomethacin in a Self-Emulsifying Controlled Release Tablet (75 mg strength) Indomethacin, a well-known Non-Steroid Anti-Inflammatory Drug (NSAID), is very popular due to the high potency of its analgesic and antiflogistic action. A side effect of indomethacin, as with most of the other NSAIDS, is the irritating effect it has on the gastric mucose. The inclusion of indomethacin (or other NSAID, e.g., diclofenac, piroxicam, naproxen, ketoprofen, etc.) in a self-emulsifying composition, may reduce the irritation caused by contact of the undissolved crystalline drug substance, with sensitive stomach and intestine mucosal surfaces.

The limited solubility of indomethacin in common oil phases required oil phase components with higher solubilization of the drug. Experimentation found that Medium Chain Tryglycerides (MCT) containing polar oils, e.g., Miglyol 812, when combined with Glycerol Monolaurate (GML) and Labrafil™1944, resulted in an oil phase component that allowed for a better rate of solubility of the drug. Tyloxapol™, a copolymer of alkylphenol and formaldehyde, was used as the pharmaceutical grade surfactant. The hydration rate of the homopolymer, polyethylene oxide (Polyox™WSR N-12K, Union Carbide) demonstrated a suitability to formation of self-emulsifying controlled release matrices. The composition of the 75 mg indomethacin self-emulsifying extended release tablet is as shown in table 4.

TABLE 4
Solid Self-Emulsifying Pharmaceutical Composition for
Sustained Delivery Of Indomethacin (75 mg)

| | Per | % |
|---|---|---|
| | tablet, | By |
| INGREDIENT | mg | Weight |
| Indomethacin | 75 | 8.85 |
| Miglyol 812 (MCT oil) | 140 | 16.53 |
| Glycerol monolaurate (GML) | 180 | 21.25 |
| Labrafil ™ 1944 | 80 | 9.45 |
| Tyloxapol ™ | 40 | 4.72 |
| Sodium Aluminium Silicate | 60 | 7.08 |
| Colloidal silicon dioxide (Aerosil ™ 300) | 40 | 4.72 |
| Lactose (spray dried) | 60 | 7.08 |
| Polyox ® WSRN 12K (Polyethylene oxide 2 min) | 100 | 11.81 |
| Microcrystalline cellulose (Avicel pH 101) | 60 | 7.08 |
| Povidone (PVP K-90) | 10 | 1.18 |
| Magnesium stearate | 2 | 0.24 |
| Tablet weight: | 847 | 100.00 |

Indomethacin, MCT oil, Labrafil 1944, glycerol monolaurate (GML) and the surfactant Tyloxapol™, were mixed together and heated to between 55 and 60° C. until a clear solution was obtained. This solution was then combined with the following sorbents: colloidal silicon dioxide, sodium aluminium silicate and a quantity of microcrystalline cellulose. The resultant paste was carefully mixed until formation of a homogenous dispersion was achieved. This dispersion was then transferred to a granulator and combined with the gel-forming polymer Polyox WSR N-12K, and a portion of the hydration rate regulator, lactose. This mixture was granulated using a separately prepared 5% binder solution of polyvinylpyrrolidone (Kollidon PVP K-90) in ethyl-alcohol until a suitable granulate was obtained. This granulate was then heated in an oven and dried at 45° C. until the volatile solvent had totally evaporated.

The resultant dried granulate was passed through 16 mesh sieve, mixed with the remaining portions of lactose, microcrystalline cellulose, and sieved magnesium stearate (lubricant). The tablets were prepared as discussed previously and had a hardness of between 2.5 and 3.5 kg.

Dissolution tests were performed in accordance with USP requirements, using USP apparatus #2 at 37° C. The paddles were rotated at 100 rpm. The tablet had completely dissolved in 6 hours. Upon dissolution, a colloidal emulsion of oil droplets had formed and was gradually released into the dissolution media, forming a hazy bluish dispersion. The active component, indomethacin, was distributed between the oil droplets and the water phase in accordance with the partition coefficient and pH of the dissolution media.

The following controlled release, self-emulsifying tablet contains 25 mg of indomethacin and was prepared in a similar manner as Example 4, using different ingredients (see Table 5).

TABLE 5
Solid Self-Emulsifying Pharmaceutical Composition for
Sustained Delivery Of Indomethacin (25 mg)

| | Per | % |
|---|---|---|
| | tablet, | By |
| INGREDIENT | mg | Weight |
| Indomethacin | 25 | 3.39 |
| Tocopherol acetate | 80 | 10.84 |
| Imwitor ™ 308 (Glycerol monocaprylate) | 80 | 10.84 |
| Mirj ® 52 | 80 | 10.84 |
| Colloidal silicon dioxide (Cab-O-Sil) | 100 | 13.55 |
| Dibasic calcium phosphate | 80 | 10.84 |
| Hydroxypropylmethylcellulose (Methocel E-50) | 80 | 10.84 |
| Lactose (spray dried) | 120 | 16.26 |
| Microcrystalline cellulose (Vivapur pH 102) | 60 | 8.13 |
| Povidone (PVP K-25) | 10 | 1.36 |
| PEG 3350 | 20 | 2.71 |
| Magnesium stearate | 3 | 0.41 |
| Tablet weight: | 738 | 100.00 |

The tablet resulting from this combination of ingredients met with suitable standards in the physical properties of hardness, friability, tabletting behavior and dissolution profile.

The sustained release delivery system of self-emulsifying compositions is capable of utilizing active ingredients of both plant and animal origin. Best results were achieved when the active ingredients were comprised of extracts.

### Example 5

Self-Emulsifying Controlled Release Tablet with 50 mg of Red Reishi Mushroom Extract Red Reishi mushrooms demonstrate high activity as an immunomodulator and have a nutritional additive. Recently, the extract of this mushroom was shown to have the capability of replacing multiple bulky doses (600 mg capsules, 3 to 4 times per day of between 20 to 50 mg) of dry material, active ingredient concentrate. The principal active ingredient components in this extract are triterpenoids, aromatic compounds and polysaccharides.

Inclusion of the Red Reishi mushroom extract in a self-emulsifying controlled release tablet significantly improved the rate of dissolution of the drug, as well as availability to the consumer. A single tablet per day provides a constant and even delivery of active ingredients to the consumer. Formed in a process consisting of dissolution of oil droplets, loaded with triterpenoids and surrounded by polysaccharides, the tablet efficiently penetrates the gastrointestine, to provide a constant supply of biologically active ingredients.

TABLE 6
Composition of a Self-Emulsifying Controlled Release
Tablet with 50 mg of Red Reishi Mushroom Extract

| | Per | % |
|---|---|---|
| | tablet, | By |
| | mg | Weight |
| Red mushrooms extract “REISHI” | 50 | 9.40 |
| Alpha-Tocopherol acetate | 25 | 4.70 |
| TPGS ™ (PEG1000 tocopherol succinate) | 25 | 4.70 |
| Colloidal silicon dioxide | 50 | 9.40 |
| Dibasic calcium phosphate | 100 | 18.80 |
| Methocel E-15 | 40 | 7.52 |
| Methocel K4M | 60 | 11.28 |
| PVP K-25 | 10 | 1.88 |
| PEG-8000 | 20 | 3.76 |
| Lactose spray dried | 100 | 18.80 |
| Microcrystalline cellulose | 50 | 9.40 |
| Magnesium stearate | 2 | 0.38 |
| Tablet weight | 532 | 100.00 |

Granulation was conducted in accordance with the protocol of example 2, but the granulate was dried at between 32 and 35° C.

The extract of the Red Reishi mushroom (Garuda Inc.), was combined with the surfactants alpha-tocopherol acetate and alpha-tocopherol acid succinate, (PEG1000 (TPGS™, Eastman)), then stirred in dry ethanol at 35° C. until a homogenous suspension was obtained. This suspension was then mixed with the sorbents, colloidal silicon dioxide and dibasic calcium phosphate. The resultant paste was carefully mixed and transferred to a suitable granulator where it was combined with methocel K4M, methocel E15 and PVP. This mixture was then granulated in ethyl-alcohol until the desired consistency of granulate was obtained. This granulate was transferred into an air-forced drying oven, and dried at a temperature of no greater than 35° C. (to prevent evaporation of the volatile, aromatic compounds of extract) until the volatile solvent had completely evaporated.

The dried granulate was then passed through a 16 mesh sieve, mixed with microcrystalline cellulose, lactose and sieved magnesium stearate (lubricant). The tablets were prepared as discussed previously, and had a hardness of between 8 and 10 kg, with a friability of less than 1%.

Dissolution time of the tablet was determined in accordance with USP 23 at 37° C., with a paddle rotation of 100 rpm, in 900 ml of water, and took approximately 6 hours using apparatus #2, at which time the tablet was more than 80% dissolved.

### Example 6

Multivitamin composition in a self-emulsifying controlled release tablet.

A formulation of both a water soluble and a lipid soluble vitamin was prepared similarly to the method described in Example 3. The composition is presented in Table 7.

TABLE 7
Self-emulsifying controlled release formulation for
water soluble and lipid soluble vitamins.

| | Per | % |
|---|---|---|
| | tablet, | By |
| INGREDIENT | mg | Weight |
| Ascorbyl palmitate (Vitamin C) | 50 | 5.88 |
| Alpha-Tocopherol acetate (Vitamin E) | 160 | 18.82 |
| Retinol acetate (Vitamin A) 10,000 I.U. | 4.5 | 0.53 |
| TPGS (Vitamin E) | 51.7 | 6.08 |
| Tocopherol acid succinate (Vitamin E) | 25 | 2.94 |
| Calcium ascorbate (Vitamin C) | 165 | 19.41 |
| Colloidal silicon dioxide | 60 | 7.06 |
| Dibasic calcium phosphate | 80 | 9.41 |
| Microcrystalline cellulose | 40 | 4.71 |
| Methocel E-15 | 60 | 7.06 |
| Methocel K4M | 20 | 2.35 |
| PVP K-25 | 10 | 1.18 |
| PEG-8000 | 20 | 2.35 |
| Lactose spray dried | 60 | 7.06 |
| Microcrystalline cellulose | 40 | 4.71 |
| Magnesium stearate | 3.8 | 0.45 |
| Tablet weight: | 850 | 100.00 |

One advantage of self-emulsifying compositions with sustained release delivery is the highly increased bio-availability of the active components contained within the composition. This sustained release delivery system is also an important factor when low solubility compounds are used, since the controlled rate of delivery can significantly decrease potentially dangerous drug saturation, and provide a constant and uniform rate of dissolution.

A further beneficial feature of self-emulsifying compositions with sustained release delivery is the entrapment of the drug within minute oil droplets that are usually less than 1 micron in diameter. This aspect leads to a significant decrease in local irritation to the lining of the gastro-intestinal tract when drugs such as NSAIDs are used. This is due to an increased efficiency in penetration of the composition through the gastrointestinal mucosal membranes which, therefore, aids in preventing a build-up of undissolved NSAID crystals on the lining of the stomach and intestine. This, in turn, eliminates the possibility of bleeding caused by the erosive action of the drug.

The composition has sufficient loading of the poorly water-soluble drug such that it provides a prolonged release of the included drug. This is due to the characteristics of the drug-bearing oil-in-water emulsion, which cause it to be released gradually from the composition. The composition is widely suitable and has high potential for use with numerous types of biologically active materials.

Additionally, sustained release of the active material allows for a change from multiple dosing (2 to 6 tablets per day), to a single dose per day. This is much more convenient for the patient and decreases the chances of dose missing, or significant variations in blood pressure level of the patient which may be caused by saturation of the drug when multiple dosing is prescribed.

Claims:

1. A composition for sustained delivery of a biologically active material, said composition comprising:
(i) a biologically active material;
(ii) a pharmaceutically acceptable hydrophobic phase for mixture with said biologically active material;
(iii) an emulsifier for providing emulsification of said hydrophobic phase in water;
(iv) a pharmaceutically acceptable sorbent; and
(v) a water absorbing polymer for providing sustained release of said biologically active material in said emulsion.

2. The composition as claimed in claim 1, wherein said hydrophobic phase comprises a compound, selected from at least one of soya oil, olive oil, kernel oil, cocoa butter, jojoba oil, fish oil.

3. The composition as claimed in claim 1, wherein said hydrophobic phase includes at least one of pharmaceutically acceptable glycerides and glycerin saturated and unsaturated fatty acid (C₂-C₂₂) esters (Medium Chain Triglycerides, tricaprin, trimyristin, triolein), mono- and diglycerides, their mixtures and derivatives (Capmul™, Miglyol™, Myvacet™, Witepsol™, Imwitor™, Dynasan™, Crodamol™).

4. The composition as claimed in claim 1, wherein said hydrophobic phase includes at least one of the group of fatty and aliphatic acid and fatty acids esters (oleic and linoleic acid, ethyl oleate, ethyl linoleate, isopropylmyristate, propyleneglycol C₂-C₁₂ esters, ethylpalmitate, isopropylpalmitate, isostearic esters, diethyladipate, diethylsebacate).

5. The composition as claimed in claim 1, wherein said hydrophobic phase includes at least one of pharmaceutical plastisizers (triethylcitrate, ethyltributylcitrate, dioctylphtalate).

6. The composition as claimed in claim 1, wherein said hydrophobic phase includes at least one of lipidic pharmaceutically acceptable compounds selected from alpha-, beta and gamma-tocopherols, tocopherol acetate, tocopherol nicotinate, retinol acetate, retinol palmitate, cholesteryl esters, stearyl alcohol and sucrose acetate isobutyrate.

7. The composition as claimed in claim 1, wherein said hydrophobic phase includes least a phospholipid compound, phosphatidylcholine, phosphatidylethanolamine, phosphatidylserine, phosphatidylinositol, phosphatidylglycerol, phosphatidic acid, sphingomyelinor a mixture thereof.

8. The composition as claimed in claim 1, wherein said surfactant, selected from group of polyoxyethylated compounds such as polyoxyethylated fatty acids, aromatic polyoxyethylated compounds, PEG-glycerides, PEG-PPG copolymers, Polyglycerines, PEG-tocopherols and propylene glycol derivatives.

9. The composition as claimed in claim 1, wherein said surfactant is selected from the group comprising sugar, polysaccharide or polyol alkyl and acyl derivatives.

10. The composition as claimed in claim 1, wherein said surfactant is selected from the group comprising anionic compounds, alkylsulfonates and amphoteric compounds.

11. The composition as claimed in claim 1, wherein said sorbent is selected from the group comprising silicon dioxide, calcium silicate, magnesium silicate and aluminium silicate, calcium phosphates, calcium sulfate.

12. The composition as claimed in claim 1, wherein said sorbent is selected from the group comprising microcrystalline cellulose, amorphous cellulose, milled cellulose, starch, crosslinked polyvinylpyrrolidone.

13. The composition as claimed in claim 1 wherein said sorbent is selected from group comprising water soluble sugars, polysaccharides and polyols.

14. The composition as claimed in claim 1, wherein said polymer is selected from group of hydroxypropylmethylcellulose, methylcellulose, hydroxyethylcellulose, hydroxypropylcellulose, caboxymethylcellulose, polyacrylic acid, alginic acid, hyaluronic acid, polygalacturonic acid, polymannuronic acid, xantan gum, locust beam gum, carrageenan, caraya gum, acacia gum, chitosan, polyethylene oxide, polyvinylpyrrolidone copolymers.

15. A process for the preparation of a composition of claim 1, comprising:
distributing said active material and a surfactant in said hydrophobic phase;
blending the mixture of said active material, said surfactant and said hydrophobic phase with at least one sorbent; and
forming a tablet.

16. The process of claim 15, wherein said active material is dissolved in said hydrophobic phase and mixed with a sorbent.

17. The process of claim 15, wherein said active material is granulated in a granulation with other components using a volatile solvent.

18. The process of claim 17, wherein granulation is prepared by the dry addition of sorbent with said hydrophobic phase and other excipients by thorough mixing.

19. The process of claim 17, wherein said granulation is prepared by compacting sorbent with said hydrophobic phase with polymers and other excipients using compactor equipment.

20. The process of claim 17, wherein said volatile solvent is selected from the group comprising methyl alcohol, ethyl alcohol, propyl alcohol, isopropyl alcohol, butyl alcohol, isobutyl alcohol, tert-butyl alcohol, acetone, methylethylketone, ethyl acetate, amylacetate, isopropyl acetate, toluene, xylol, metylene chloride, trichlormethane, tetrachlormethane, methane, dichloroethane, purified water and water-alcohol mixtures.
